(19) United States
(12) Patent Application Publication
(10) Pub. No.: US 2011/0288331 A1
(43) Pub. Date: Nov. 24, 2011

(54) PROCESS FOR THE PREPARATION OF CHOLINE SALT OF FENOFIBRIC ACID AND ITS NOVEL POLYMORPH

(75) Inventors: Ravi Ponnaiah, Vadodara (IN); Sanjay Desai, Vadodara (IN); Dhiraj Rathod, Vadodara (IN); Lalit Katariya, Vadodara (IN); Nilesh Bhimani, Vadodara (IN); Viral Modi, Vadodara (IN)

(73) Assignee: Alembic Pharmaceuticals Limited, Vadodara (IN)

(21) Appl. No.: 13/146,031

(22) PCT Filed: Dec. 28, 2009
(86) PCT No.: PCT/IB2009/055959

§ 371 Date: Jul. 25, 2011

(30) Foreign Application Priority Data
Jan. 30, 2009 (IN) 187/MUM/2009

(51) Int. Cl. C07C 51/41; C07C 51/42; C07C 59/84

(52) U.S. Cl. 562/460

(57) ABSTRACT

The present invention relates to an improved process for the preparation of choline salt of fenofibric acid corresponding to formula (I). The present invention also provides crystalline polymorphic form of choline salt of fenofibric acid corresponding to formula (I) designated as form A.

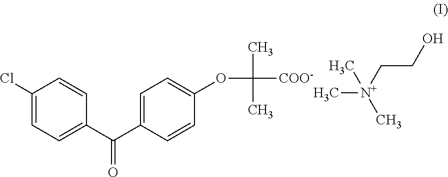

### FIELD OF THE INVENTION

The present invention relates to an improved process for the preparation of choline salt of fenofibric acid corresponding to formula (I). The present invention also provides crystalline polymorphic form of choline salt of fenofibric acid corresponding to formula (I) designated as form A.

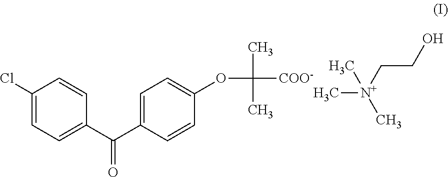

### BACKGROUND OF THE INVENTION

Fenofibrate of formula (IV) belongs to class of fibrate drugs. It is useful to reduce both low-density lipoprotein (LDL) and very low density lipoprotein (VLDL) levels, as well as increasing high-density lipoprotein (HDL) levels and reducing triglycerides level. It also has a beneficial effect on the insulin resistance featured by the metabolic syndrome. Fenofibrate can be used alone or in conjunction with statins in the treatment of hypercholesterolemia and hypertriglyceridemia.

Fenofibric acid of formula (Ia) is the active metabolite of fenofibrate, which also produces reductions in total cholesterol, LDL cholesterol, apolipoprotein B, total triglycerides and triglyceride rich lipoprotein (VLDL) in treated patients.

Fenofibrate and its acid were first disclosed in U.S. Pat. No. 4,058,552. The process for synthesis of Fenofibrate as disclosed in this patent is as follows:

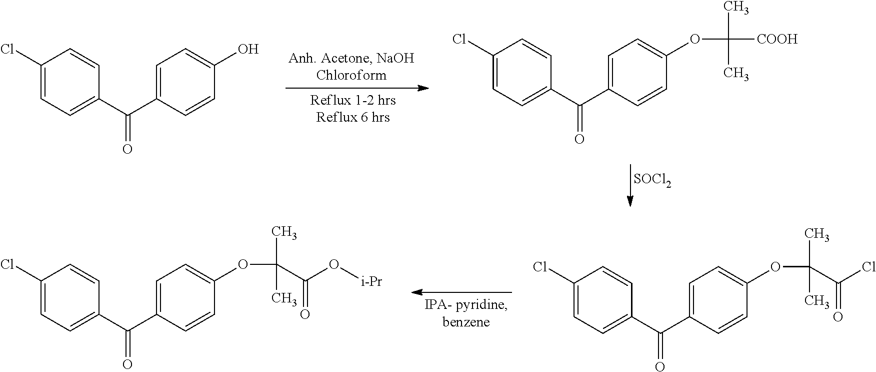

Further U.S. Pat. No. 4,179,515 describes process for preparation of Fenofibrate which is as follows:

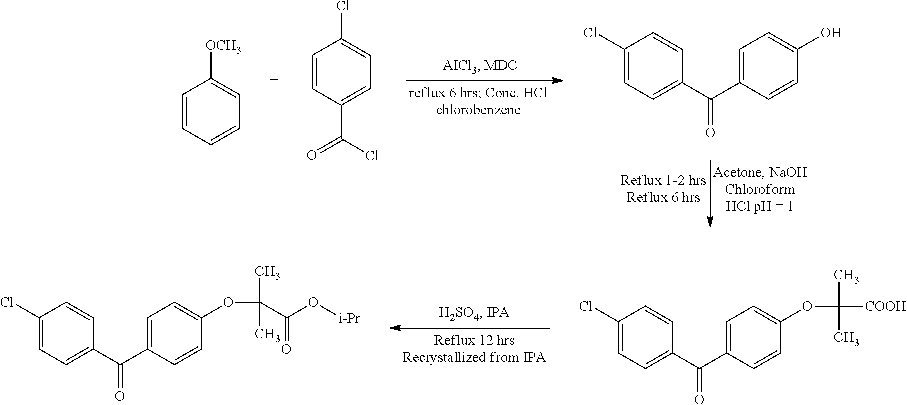

The choline salt of Fenofibric acid is first disclosed and claimed in U.S. Pat. No. 7,259,186. The process for preparation disclosed in this patent is as follows:

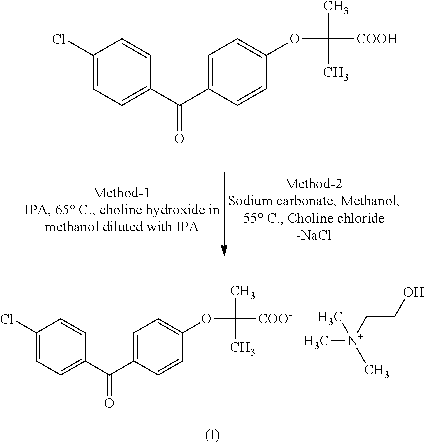

Method-2 as shown hereinabove involves reaction of Fenofibric acid with choline chloride in presence of sodium carbonate and water. Here, sodium chloride is obtained as bi-product which precipitates out from reaction mixture. Sodium chloride is filtered off from reaction mass. However, sodium chloride being partially soluble in methanol passes alongwith the fenofibric acid choline salt and thereby contaminating the final product.

US patent application no. 20080275270 describes process for preparation of fenofibric acid choline salt which is as follows:

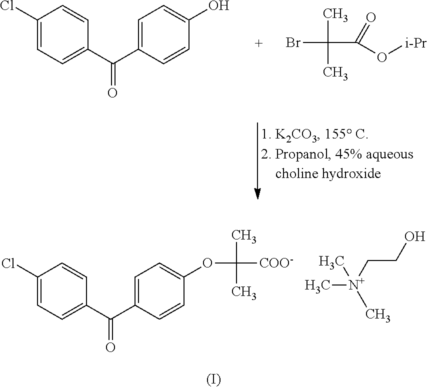

This process involves reaction of (4-chlorophenyl)(4-hydroxyphenyl)methanone with isopropyl-2-bromo-2-methylpropanote in presence of potassium carbonate to give an intermediate which is in single operation converted to choline salt of fenofibric acid using choline hydroxide. The overall yield of the reaction is 67-70%.

In light of above mentioned prior arts, there exits a need to develop a process for preparation of Fenofibric acid choline salt which results in product without undesired impurities.

Further, polymorphic forms of choline salt of Fenofibric acid are neither disclosed nor characterized in any reference till date. Polymorphism is the occurrence of different crystalline forms of a single compound and it is a property of some compounds and complexes in solid state. The polymorphic and pseudopolymorphic solids display different physical properties, including those due to packing, and various thermodynamic, spectroscopic, interfacial and mechanical properties (See H. Brittain, Polymorphism in Pharmaceutical Solids, Marcel Dekker, New York, N.Y., 1999, pp. 1-2).

The inventor of present invention have developed and characterized novel polymorph of choline salt of Fenofibric acid designated as Form A.

### OBJECT OF THE INVENTION

An object of the present invention is to provide an improved process for the preparation of choline salt of fenofibric acid corresponding to formula (I).

Another object of the present invention is to provide novel polymorph of choline salt of fenofibric acid corresponding to formula (I) designated as Form A.

Yet another object of the present invention is to provide a process for preparation of choline salt of fenofibric acid corresponding to formula (I) which offers advantage over existing prior art process.

### SUMMARY OF THE INVENTION

An aspect of the present invention provides an improved process for the preparation of choline salt of fenofibric acid corresponding to formula (I).

Another aspect of the present invention provides an improved process for the preparation of choline salt of fenofibric acid corresponding to formula (I) comprising of reacting fenofibric acid of formula (II) with choline chloride in presence of organic base and suitable solvent.

Yet another object of the present invention provides novel polymorph of choline salt of fenofibric acid corresponding to formula (I) designated as Form A.

### BRIEF DESCRIPTION OF THE DRAWINGS

FIG. 1: PXRD pattern of Form A of choline salt of fenofibric acid corresponding to formula (I)

### DETAILED DESCRIPTION OF THE INVENTION

The present invention provides an improved process for the preparation of choline salt of fenofibric acid corresponding to formula (I) comprising of reacting fenofibric acid of formula (II) with choline chloride in presence of organic base and suitable solvent.

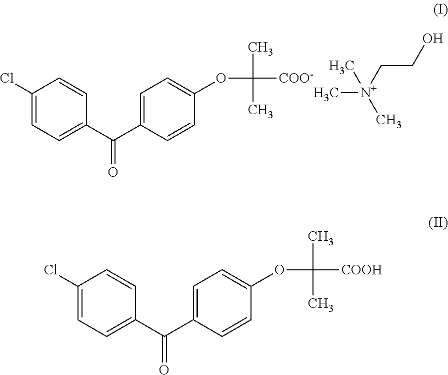

The Fenofibric acid used for process of present invention can be prepared by any method known perse.

Fenofibric acid of formula (II) is reacted with choline chloride in presence of organic base. The organic base can be selected from group comprising of NR¹R²R³, wherein R¹, R², R³ are independently H or C₁₋₄ straight or branched alkyl, morpholine, dimethylaniline, pyridine, piperidine, N-methylpyrrolidine, N-methylpyrrolidone and the like or mixtures thereof. Examples of NR¹R²R³ include but are not limited to dimethylamine, triethylamine, diethylamine, tert-butylamine and the like.

The suitable solvent can be selected from alcoholic solvent selected from group comprising of methanol, ethanol, n-butanol, isopropanol and the like or mixtures thereof.

The reaction is carried out preferably at room temperature or at reflux temperature of the solvent.

After completion of the reaction, the reaction mixture is cooled to about 0-5° C. The solid obtained is isolated by conventional methods. The bi-product formed during the reaction which is hydrochloride salt of organic base used in the reaction is highly soluble in the organic solvent and passes completely into the filtrate. Thereby, the product obtained is free from undesired impurity.

If desired the obtained choline salt of fenofibric acid is treated with suitable solvent at about 0° C. to about ambient temperature and isolated by conventional method. Treating involves suspending, leaching or making slurry.

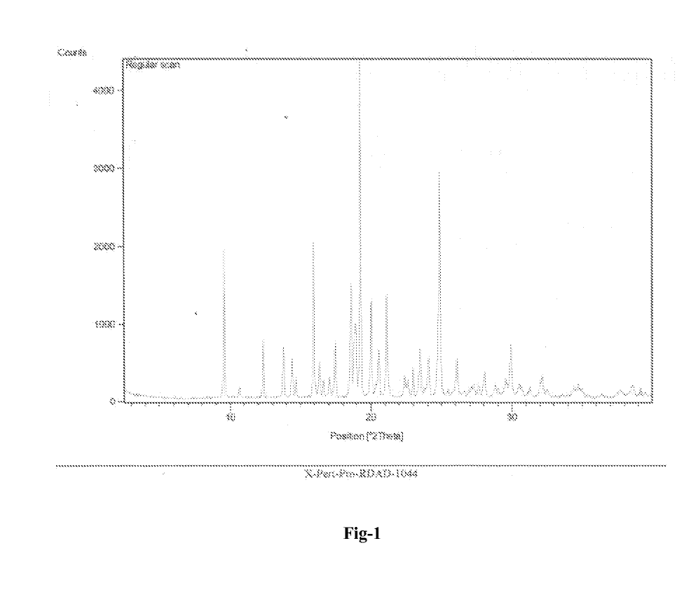

Further, aspect of present invention provides novel polymorph of choline salt of fenofibric acid corresponding to formula (I) designated as Form A. The polymorph was characterized by PXRD pattern substantially similar to that disclosed in FIG. 1.

The instrument used for scanning the sample for PXRD is PAN analytical, X-Pert-Pro-RDAD-1044.

Form A of choline salt of fenofibric acid corresponding to formula (I) has an x-ray powder diffractogram having characteristic peaks expressed as 20 values at about 9.61, 15.96, 19.27, 24.89±0.2°θ.

A preferred embodiment of the present invention provides process for preparation of Form A of choline salt of fenofibric acid corresponding to formula (I) comprising steps of

(a) reacting fenofibric acid of formula (II) with choline chloride in presence of organic base and suitable solvent
(b) cooling the reaction mixture at about 0° C. to about ambient temperature
(c) isolating Form A

The process of the present invention is described by the following examples, which are illustrative only and should not be construed so as to limit the scope of the invention in any manner.

### Example-1

### Preparation of Choline Salt of Fenofibric Acid

Charge 10 gm fenofibric acid and 90 ml absolute ethanol and 4.5 ml triethylamine in to 250 ml RBF. Stir it for 10-15 minutes to get clear solution. Charge in to 250 ml RBF and charge 4.4 gm choline chloride. Heat it up to reflux temperature to get clear solution. Filter it through hyflo bed and wash it with absolute ethanol (10 ml). Cool it up to ambient temperature. Stir it for 12-15 hours at ambient temperature. Cool it at 0-5° C. and stir for 2 hours at 0-5° C. Filter it and suck dry it. Charge wet cake in to 250 ml RBF and 50 ml absolute ethanol. Stir it for one hour at ambient temperature. Filter it and wash it with 10 ml absolute ethanol. Dry it at 50-60° C. Dry wt.—8.0 gm (yield: ˜75%).

We claim:

1. An improved process for the preparation of choline salt of fenofibric acid corresponding to formula (I) comprising of reacting fenofibric acid of formula (II) with choline chloride in presence of organic base and suitable solvent

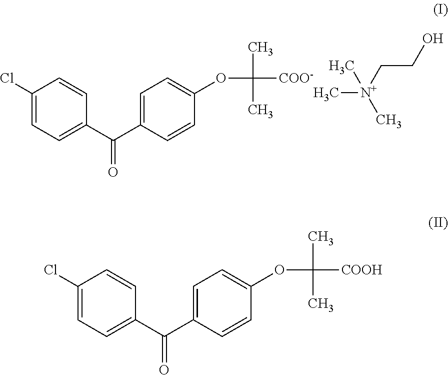

2. A process as claimed in claim 1, wherein said organic base can be selected from group comprising of NR¹R²R³, wherein R¹, R², R³ are independently H or C₁₋₄ straight or branched alkyl, morpholine, dimethylaniline, pyridine, piperidine, N-methylpyrrolidine, N-methylpyrrolidone and mixtures thereof.

3. A process as claimed in claim 2, wherein said NR¹R²R³ is selected from group comprising of dimethylamine, triethylamine, diethylamine and tert-butylamine.

4. A process as claimed in claim 1, wherein said suitable solvent is selected from group comprising of methanol, ethanol, n-butanol, isopropanol or mixtures thereof.

5. Form A of choline salt of fenofibric acid corresponding to formula (I) having an x-ray powder diffractogram having characteristic peaks expressed as 2θ values at about 9.61, 15.96, 19.27, 24.89±0.2°θ.

6. A process for preparation of Form A of choline salt of fenofibric acid corresponding to formula (I) comprising steps of
(a) reacting fenofibric acid of formula (II) with choline chloride in presence of organic base and suitable solvent
(b) cooling the reaction mixture at about 0° C. to about ambient temperature
(c) isolating Form A

7. A process as claimed in claim 6, wherein said organic base can be selected from group comprising of NR¹R²R³, wherein R¹, R², R³ are independently H or C₁₋₄ straight or branched alkyl, morpholine, dimethylaniline, pyridine, piperidine, N-methylpyrrolidine, N-methylpyrrolidone and mixtures thereof.

8. A process as claimed in claim 7, wherein said NR¹R²R³ is selected from group comprising of dimethylamine, triethylamine, diethylamine and tert-butylamine.

9. A process as claimed in claim 6, wherein said suitable solvent is selected from group comprising of methanol, ethanol, n-butanol, isopropanol or mixtures thereof.

10. A process as claimed in claim 6, which further comprises treating with suitable solvent at about 0° C. to about ambient temperature.
